(19) United States
(12) Patent Application Publication
(10) Pub. No.: US 2023/0130357 A1
(43) Pub. Date: Apr. 27, 2023

(54) MULTI-LAYER HERNIA MESHES AND METHODS OF MANUFACTURE AND USE THEREOF

(71) Applicant: BOARD OF REGENTS OF THE UNIVERSITY OF NEBRASKA, Lincoln, NE (US)

(72) Inventors: Mark A. CARLSON, Omaha, NE (US); Jingwei XIE, Omaha, NE (US)

(21) Appl. No.: 17/918,024

(22) PCT Filed: Apr. 30, 2021
(86) PCT No.: PCT/US2021/030083

§ 371 Date: Oct. 10, 2022

Related U.S. Application Data
(60) Provisional application No. 63/017,735, filed on Apr. 30, 2020.

(51) Int. Cl. A61F 2/00; A61L 31/04; A61L 31/16; A61L 31/10

(52) U.S. Cl. CPC A61F 2/0063; A61L 31/041; A61L 31/16; A61L 31/10; A61F 2240/001; A61L 2400/12; A61F 2210/0076

(57) ABSTRACT

Hernia meshes are provided as well as methods of use thereof and methods of making.

This application claims priority under 35 U.S.C. § 119(e) to U.S. Provisional Patent Application No. 63/017,735, filed Apr. 30, 2020. The foregoing application is incorporated by reference herein.

### FIELD OF THE INVENTION

This application relates to the fields of hernia meshes. More specifically, this invention provides methods of synthesizing hernia meshes and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Approximately ˜300,000 inpatient hernia repairs are performed in the U.S. each year. Most of these repairs are accomplished with implantation of a synthetic non-resorbable (permanent) mesh, such as polypropylene (Carlson, et al. (2008) Hernia 12(1):9-22; Brown, et al. (2010) Ann. R. Coll. Surg. Engl., 92(4):272-278; Nguyen, et al. (2014) JAMA Surg., 149(5):415-421; Bringman, et al. (2010) Hernia 14(1):81-87; Bilsel, et al. (2012) Int. J. Surg., 10(6):317-321; Schumpelick, et al. (2010) Hernia Repair Sequelae, Berlin: Springer; Huerta, et al. (2016) JAMA Surg., 151(4):374-381). In 5-10% of cases, however, permanent hernia mesh is contraindicated because of the presence of infection, gross enteric contamination, severe inflammation, proximity to hollow viscera, and other reasons (Brown, et al. (2010) Ann. R. Coll. Surg. Engl., 92(4):272-278; Bringman, et al. (2010) Hernia 14(1):81-87; Bilsel, et al. (2012) Int. J. Surg., 10(6):317-321; Schumpelick, et al. (2010) Hernia Repair Sequelae, Berlin: Springer; Chand, et al. (2012) Hernia 16(2):223-226). In most of these complex hernia cases, a biologic mesh (e.g., processed human cadaver or porcine skin) is utilized for the repair (Huerta, et al. (2016) JAMA Surg., 151(4):374-381; Rosen, et al. (2013) Ann. Surg., 257(6):991-996). Biologic mesh is expensive (30-50 USD/cm², as compared with 0.10 USD/cm² for simple permanent mesh) and has questionable long-term efficacy (Rosen, et al. (2017) Ann. Surg., 265(1):205-211; Primus, et al. (2013) Hernia 17(1):21-30; Harris, H. W. (2013) Ann. Surg., 257(6):997-998; Huntington, et al. (2016) Surgery 160(6):1517-1527). Currently, there are four synthetic fully-resorbable hernia meshes available in the United States: (i) Vicryl™ Mesh (polyglactin 910; Ethicon Inc.); (ii) BIO-A® Tissue Reinforcement (polyglycolic acid & trimethylene carbonate; W.L. Gore & Associates, Inc.); (iii) Phasix™ Mesh (poly-4-hydroxybutyrate; Bard Davol Inc.); and (iv) TIGR Matrix™ (copolymer of glycolide, lactide and trimethylene carbonate; Novus Scientific, Inc.). Vicryl™ mesh has not been effective for abdominal wall repair and the other three are relatively recent additions to the hernia mesh market without a provien track record and are priced nearly as expensive as biologic meshes (Shankaran, et al. (2011) Ann. Surg., 253(1):16-26). Accordingly, there is a need for a synthetic, slowly-resorbable hernia mesh that is cheaper and more efficacious compared to biologic mesh for use in the repair of complicated hernia and also for incisional support in obese patients, who have a higher risk of hernia.

### SUMMARY OF THE INVENTION

In accordance with the instant invention, hernia meshes and methods of producing the hernia meshes are provided. The hernia mesh of the instant invention can be synthesized in any shape and are broadly configurable (e.g., with different membranes of different properties). The hernia mesh may be non-resorbable, at least partially resorbable, or completely resorbable. In certain embodiments, the hernia mesh comprising multiple layers selected from 2D nanofiber membranes, 3D expanded nanofiber membranes, and/or strengthening or support membranes. In certain embodiments, the hernia mesh comprises a 2D nanofiber membrane and a 3D expanded nanofiber membrane and, optionally, a strengthening or support membrane. In certain embodiments, the hernia mesh comprises one layer of a 3D expanded nanofiber membrane, one layer of a strengthening or support membrane, and a second layer of a 3D expanded nanofiber membrane. In certain embodiments, the 2D nanofiber membranes and the 3D expanded nanofiber membranes comprise polycaprolactone (PCL) and polylactide (PLA). In certain embodiments, the 2D nanofiber membranes and the 3D expanded nanofiber membranes comprise electrospun nanofibers. The 3D expanded nanofiber membrane may be manufactured using a gas expansion method. For example, the 3D expanded nanofiber membrane may be expanded by exposure to a subcritical fluid such as subcritical CO₂ and then depressurized. In certain embodiments, the 2D nanofiber membrane and/or 3D expanded nanofiber membrane comprises one or more active agents such as, without limitation, antimicrobial agents, chemokines, cytokines, and growth factors. In certain embodiments, the surface of the 3D expanded nanofiber membrane comprises extra cellular matrix mimetic materials, a hydrogel, antimicrobial agents, and/or growth factors. The 3D expanded nanofiber membrane may also comprise a material that enhances water absorption, such as gelatin, chitosan, or collagen. In certain embodiments, the 3D expanded nanofiber membrane is crosslinked. In certain embodiments, the 3D expanded nanofiber membrane is coated with gelatin and crosslinked. Methods for producing the hernia mesh are also provided.

In accordance with another aspect of the instant invention, methods of using the hernia mesh are provided. For example, the hernia mesh may be used to reduce, inhibit, prevent, and/or treat a hernia in a subject.

### BRIEF DESCRIPTION OF THE DRAWINGS

FIG. 1A provides an image of the 2D side of a fully resorbable mesh comprising a sheet of 2D non-expanded electrospun blend of PCL:PLA (50:50) bonded to a sheet of 3D expanded electrospun blend of PCL:PLA (50:50). FIG. 1B provides an image of the 3D side of the fully resorbable mesh provided in FIG. 1A. FIG. 1C provides a side view of the fully resorbable mesh provided in FIG. 1A, showing the relative thickness of each layer. FIG. 1D provides an image of the 3D side of a partially resorbable mesh comprising a sheet of 2D non-expanded electrospun blend of PCL:PLA (50:50), a sheet of 3D expanded electrospun blend of PCL:PLA (50:50), and a sheet of lightweight polypropylene sandwiched between the 2D and 3D sheets.

FIG. 2 shows the incision location for the porcine hernia model. The ventral surface of the pig is shown. Large black arrow is cephalad. Dashed line 1 is location of upper midline incision. Dashed line 2 is location of central midline incision. UMM=urethral meatus of male. UMF=urethral meatus of female. Dashed ovel is approximate location of male urethra.

FIG. 3 provides a schematic of the intraperitoneal placement of mesh (IPOM). A cross section of the human abdomen is depicted. Mesh is place directly underneath abdominal wall (AW) to buttress the surgical incision (SI), with viscera (V) directly underneath. SSC=skin & subcutaneous tissue.

FIG. 4A provides an image of the porcine abdominal cavity reopened at necropsy at 90 days. The pig was treated with the fully resorbable mesh. FIG. 4B provides an image of the porcine abdominal cavity reopened at necropsy. The pig was treated with the partially resorbable mesh.

### DETAILED DESCRIPTION OF THE INVENTION

The present invention describes compositions and methods for the treatment, prevention, and/or repair of hernias. The compositions of the present invention comprise a multi-layer hernia mesh. The multi-layer hernia mesh is broadly configurable and can be varied to have the desired tensile strength, thickness, mesh area and/or shape, rate of absorption, and/or active agents delivered. The multi-layer hernia mesh and/or individual membranes/layers thereof may be non-resorbable, at least partially resorbable, or fully resorbable. For example, the addition of a non-resorbable polymer (e.g., polypropylene) within one or more membranes/layers can make the membrane only partially absorbable with enduring tensile strength.

In certain embodiments, the multi-layer hernia mesh comprises a first layer comprising at least one two-dimensional (2D) (or non-expanded) nanofiber membrane (or mat) and at least one three-dimensional (3D) expanded nanofiber membrane (or structure). The expanded nanofiber membrane may also be referred to as a 3D porous scaffold or 3D scaffold. The 3D expanded nanofiber membranes may be porous scaffolds, inverse opal scaffolds (e.g., porous structures with an ordered array of uniform pores with dimensions on the nano- and micrometer scale), porous or expanded scaffolds generated by supercritical CO₂ fluid, porous or expanded scaffolds generated by gas generation, nanofiber aerogels, porous scaffolds generated by freeze drying, porous scaffolds generated by 3D printing, porous scaffolds generated by phase separation, or porous scaffolds generated by using sacrificial template. In certain embodiments, the multi-layer hernia mesh further comprises at least one strengthening or support layer (e.g., an approved, non-resorbable synthetic material). For example, the strengthening or support layer may be placed between the 2D nanofiber membrane and the 3D expanded nanofiber membrane.

The nanofibers and nanofiber membranes of the instant invention can be fabricated by any method. For example, the 2D nanofiber membranes of the present invention can be made using a variety of manufacturing techniques including, but not limited to, weaving, knitting, electrospraying, and electrospinning. In a particular embodiment, the nanofiber membranes comprise electrospun nanofibers. The nanofiber membranes may comprise aligned fibers (e.g., uniaxially aligned), random fibers, and/or entangled fibers. In a particular embodiment, the nanofiber membranes comprise aligned fibers (e.g., uniaxially, radially, vertically, or horizontally). While the application generally describes nanofiber (fibers having a diameter less than about 1 μm (e.g., average diameter)) membranes and the synthesis of three-dimensional expanded nanofibrous structures, the instant invention also encompasses microfiber (fibers having a diameter greater than about 1 μm (e.g., average diameter)) membranes and the synthesis of three-dimensional microfibrous structures.

The methods of the instant invention may further comprise synthesizing the nanofiber membranes prior to expansion (e.g., exposure to gas bubbles). In a particular embodiment, the nanofiber membrane is synthesized using electrospinning. In a particular embodiment, the nanofiber membrane comprises aligned fibers (e.g., uniaxially), random fibers, and/or entangled fibers. The nanofiber membrane may be cut or shaped prior to expansion. In a particular embodiment, the nanofiber membrane is cut or shaped under cryogenic or frozen conditions (e.g., in liquid nitrogen). The nanofiber membrane can be cut or shaped into any desired shape such as, without limitation: rectangles, squares, triangles, quadrangles, pentagons, hexagons, circles, ovals, semicircles, L's, C's, O's, U's, and arches. In certain embodiments, the nanofiber membrane is cut to a size appropriate to cover the hernia or for incision support.

In certain embodiments, the nanofiber membrane is expanded or thickened into an expanded nanofiber membrane by exposing the nanofiber membrane to gas bubbles. The bubbles can be generated by chemical reactions or physical manipulations. For example, the nanofiber membrane can be submerged or immersed in a bubble/gas producing chemical reaction or physical manipulation. Generally, the longer the exposure to the bubbles, the greater the thickness and porosity of the expanded nanofiber membrane increases. The nanofiber membrane may also be expanded within a mold (e.g., a metal, plastic, or other material that does not expand in the presence of gas bubbles) to assist in the formation of a desired shape. The nanofiber membrane may be treated with air plasma prior to exposure to gas bubbles (e.g., to increase hydrophilicity).

After exposure to the bubbles, the expanded nanofiber membrane may be washed and/or rinsed in water and/or a desired carrier or buffer (e.g., a pharmaceutically or biologically acceptable carrier). Trapped gas bubbles may be removed by applying a vacuum to the expanded nanofiber membrane. For example, the expanded nanofiber membrane may be submerged or immersed in a liquid (e.g., water and/or a desired carrier or buffer) and a vacuum may be applied to rapidly remove the gas bubbles. After expansion (e.g., after rinsing and removal of trapped gas), the expanded nanofiber membrane may be placed in storage in cold solution or lyophilized and/or freeze-dried.

The gas bubbles of the instant invention can be made by any method known in the art. The bubbles may be generated, for example, by chemical reactions or by physical approaches. Electrospun nanofiber membranes can be expanded in the third dimension with ordered structures using gas bubbles generated by chemical reactions in an aqueous solution (see, e.g., WO 2016/053988; WO 2019/060393; Jiang et al. (2018) Acta Biomater., 68:237-248; Jiang, et al. (2015) ACS Biomater. Sci. Eng., 1:991-1001; Jiang, et al. (2016) Adv. Healthcare Mater., 5:2993-3003; Joshi, et al. (2015) Chem. Eng. J., 275:79-88; each of the foregoing incorporated by reference herein). In a particular embodiment, the chemical reaction or physical manipulation does not damage or alter or does not substantially damage or alter the nanofibers (e.g., the nanofibers are inert within the chemical reaction and not chemically modified). As explained hereinabove, the nanofiber membrane may be submerged or immersed in a liquid comprising the reagents of the bubble-generating chemical reaction. Examples of chemical reactions that generate bubbles include, without limitation:

NaBH₄+2H₂O=NaBO₂+4H₂

NaBH₄+4H₂O=4H₂(g)+H₃BO₃+NaOH

HCO₃⁻+H⁺=CO₂+H₂O

NH₄⁺+NO₂⁻=N₂+2H₂O

H₂CO₃=H₂O+CO₂

2H⁺+S²⁻=H₂S

2H₂O₂=O₂+2H₂O

3HNO₂=2NO+HNO₃+H₂O

HO₂CCH₂COCH₂CO₂H=2CO₂+CH₃COCH₃

2H₂O₂=2H₂+O₂

CaC₂+H₂O=C₂H₂

Zn+2HCl=H₂+ZnCl₂

2KMnO₄+16HCl=2KCl+2MnCl₂+H₂O+SCl₂

In a particular embodiment, the chemical reaction is the hydrolysis of NaBH₄ (e.g., NaBH₄+2H₂O=NaBO₂+4H₂). In a particular embodiment, CO₂ gas bubbles (generated chemically or physically) are used (e.g., for hydrophilic polymers).

Examples of physical approaches for generating bubbles of the instant invention include, without limitation: 1) create high pressure (fill gas)/heat in a sealed chamber and suddenly reduce pressure; 2) dissolve gas in liquid/water in high pressure and reduce pressure to release gas bubbles; 3) use supercritical fluids (reduce pressure) like supercritical CO₂; 4) use subcritical gas liquid (then reduce pressure) (e.g., liquid CO₂, liquid propane and isobutane); 5) fluid flow; 6) apply acoustic energy or ultrasound to liquid/water; 7) apply a laser (e.g., to a liquid or water); 8) boiling; 9) reduce pressure boiling (e.g., with ethanol); and 10) apply radiation (e.g., ionizing radiation on liquid or water). The nanofiber membrane may be submerged or immersed in a liquid of the bubble-generating physical manipulation.

In a particular embodiment, the nanofiber membranes are expanded using a subcritical or supercritical fluid or liquid (e.g., CO₂, N₂, N₂O, hydrocarbons, and fluorocarbons). In a particular embodiment, liquid CO₂ is utilized. For example, nanofiber membranes may be expanded by exposing to, contacting with or being placed into (e.g., submerged or immersed) a subcritical liquid/fluid (e.g., subcritical CO₂) and then depressurized. The cycle of placing the nanofibrous structures into subcritical CO₂ and depressurizing may be performed one or more times. Generally, the more times the expansion method is used the thickness and porosity of the nanofibrous (or microfibrous) membrane increases. For examples, the cycle of exposure to subcritical CO₂ and then depressurization may be performed one, two, three, four, five, six, seven, eight, nine, ten, or more times, particularly 1-10 times, 1-5 times, or 1-3 times. In a particular embodiment, the cycle of exposure to subcritical CO₂ and then depressurization is performed at least 2 times (e.g., 2-10 times, 2-5 times, 2-4 times, or 2-3 times). In a particular embodiment, the method comprises placing the nanofibrous membrane and dry ice (solid CO₂) in a sealed container, allowing the dry ice to turn into liquid CO₂, and then unsealing the container to allow depressurization.

The nanofiber membrane and subcritical fluid (e.g., subcritical CO₂; or solid form of subcritical fluid (e.g., dry ice)) may be contained in any suitable container (e.g., one which can withstand high pressures). For example, the subcritical fluids and the nanofiber membrane may be contained within, but not limited to: chambers, vessels, reactors, chambers, and tubes. In a particular embodiment, the equipment or container used during the methods of the present invention will have a feature or component that allows control of the depressurization rate of the subcritical fluid. Depressurization of the subcritical fluid can be done using a variety of methods including but not limited to manually opening the container to decrease pressure or by using some type of equipment that can regulate the rate of depressurization of the reaction vessel.

The nanofibers of the instant invention may comprise any polymer. In a particular embodiment, the polymer is biocompatible and/or non-toxic. The polymer may be biodegradable or non-biodegradable. In a particular embodiment, the polymer is a biodegradable polymer. The polymer may by hydrophobic, hydrophilic, or amphiphilic. In a particular embodiment, the polymer is hydrophobic. In a particular embodiment, the polymer is hydrophilic. The polymer may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene).

Examples of hydrophilic polymers include, without limitation: polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment) and a hydrophobic polymer (e.g., segment) from those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

Examples of polymers particularly useful for electrospinning are provided in Xie et al. (Macromol. Rapid Commun. (2008) 29:1775-1792; incorporated by reference herein; see e.g., Table 1). Examples of compounds or polymers for use in the fibers of the instant invention, particularly for electrospun nanofibers include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/gelatin, PCL/collagen, sodium aliginate/PEO, chitosan/PEO, Chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan), and composites (e.g., PDLA/HA, PCL/CaCO₃, PCL/HA, PLLA/HA, gelatin/HA, PCL/collagen/HA, collagen/HA, gelatin/siloxane, PLLA/MWNTs/HA, PLGA/HA). In certain embodiments, the nanofiber membrane and expanded nanofiber membrane may be made from a variety of polymers comprising, but not limited to: polymethacrylate, poly vinyl phenol, polyvinylchloride, cellulose, polyvinyl alcohol, polyacrylamide, PLGA, collagen, polycaprolactone, polyurethanes, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene glycol, poly(ethylene-co-vinyl alcohol), polyacrylnitrile, polyvinyl pyrrolidone, polymetha-phenylene isophthalamide, alginate, gelatin, chitosan, starch, pectin, cellulose, methylcellulose, sodium polyacrylate, starch-acrylonitrile co-polymers, and/or combinations of two or more polymers.

In a particular embodiment, the polymer comprises polycaprolactone (PCL). In a particular embodiment, the polymer comprises polycaprolactone (PCL) and polylactide (PLA) (e.g., at a 1:1 ratio). In a particular embodiment, the nanofiber membrane and/or expanded nanofiber membrane further comprises polypropylene.

In a particular embodiment, the nanofiber membrane and/or expanded nanofiber membrane may further comprise at least one amphiphilic block copolymer comprising hydrophilic poly(ethylene oxide) (PEO) and hydrophobic poly(propylene oxide) (PPO). In a particular embodiment, the nanofiber membrane and/or expanded nanofiber membrane comprises a poloxamer or an amphiphilic triblock copolymer comprising a central hydrophobic PPO block flanked by two hydrophilic PEO blocks (i.e., an A-B-A triblock structure). In a particular embodiment, the amphiphilic block copolymer is selected from the group consisting of Pluronic® L31, L35, F38, L42, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R4, 17R8, 22R4, 25R1, 25R2, 25R4, 25R5, 25R8, 31R1, 31R2, and 31R4. In a particular embodiment, the nanofiber membrane and/or expanded nanofiber membrane comprises poloxamer 407 (Pluronic® F127). The amphiphilic block copolymer (e.g., poloxamer) may be added in various amounts to the polymer solution during the synthesis process (e.g., electrospinning). In a particular embodiment, 0% to 20%, particularly 0% to 10%, of the polymer solution is amphiphilic block copolymer (e.g., poloxamer). In a particular embodiment, 0.1% to 5%, particularly 0.5% to 2%, of the polymer solution is amphiphilic block copolymer (e.g., poloxamer).

The strengthening or support membrane of the hernia mesh may comprise an approved, non-resorbable synthetic material. For example, the strengthening or support membrane of the hernia mesh may be made from a variety of materials including but not limited to polypropylene (e.g., light-weight or medium-weight polypropylene mesh), polyester, or other biologically permanent, nonresorbable material. In a particular embodiment, the polymer of the strengthening or support membrane is biocompatible and/or non-toxic. In a particular embodiment, the polymer of strengthening or support membrane is non-biodegradable. When present, the strengthening or support membrane provides long-term tensile strength to the mesh. In certain embodiments, the strengthening or support membrane is incorporated within the 2D membrane, incorporated between the 2D and 3D membranes, or otherwise incorporated into the mesh so that it is not in direct contact with the intraabdominal viscera.

As explained herein, the 2D nanofiber membrane provides a physical barrier between the hollow viscera (e.g., small intestines) or other internal organs and the rest to the mesh components. In a particular embodiment, this 2D nanofiber membrane is relatively resistant to tissue and cell ingrowth. The 2D nanofiber membrane may be non-resorbable, partially resorbable, or resorbable. In certain embodiment, the 2D nanofiber membrane may comprise surface modifications (e.g., antifouling materials such as poly(ethylene glycol) (PEG), or zwitterionic materials (e.g., zwitterionic polymers, poly(sulfobetaine methacrylate) (PSBMA), poly(carboxybetaine methacrylate) (pCBMA), or those described in WO 2020/226622 and US Patent Application Publication No. 2020/0308440, each incorporated herein by reference)) to discourage adhesion formation with intraabdominal viscera. In a typical hernia repair in which the mesh is located within the peritoneal cavity (e.g., underlay reinforcement of a hernia repair), the 2D nanofiber membrane is positioned so that it is facing the intraabdominal viscera.

As explained herein, the 3D expanded nanofiber membrane may encourage ingrowth of cells and tissues resulting in mesh incorporation and anchorage into the tissue. This action promotes a durable repair over the long term. The 3D expanded nanofiber membrane may be non-resorbable, partially resorbable, or resorbable. In a typical hernia repair, the 3D expanded nanofiber membrane is placed so that it is facing the abdominal wall.

The 2D nanofiber membrane and/or 3D expanded nanofiber membrane may be modified with or contain active agents (e.g., therapeutic compounds) including but not limited to antimicrobial agents (e.g., antibiotics, silver ions, antimicrobial peptides), cytokines, chemokines, and/or growth factors (e.g., PDGF, VEGF, EGF). The active agents may be incorporated into the 2D nanofiber membrane and/or 3D expanded nanofiber membrane such that they are slowly released overtime. More than one active agent may be incorporated into a 2D nanofiber membrane and/or 3D expanded nanofiber membrane. The surface of the 2D nanofiber membrane and/or 3D expanded nanofiber membrane can also be readily modified with extra cellular matrix mimetic materials such as, without limitation: fibronectin, collagen, laminin, elastin, etc. and/or other antimicrobial agents and/or growth factors. In certain embodiments, at least the 3D expanded nanofiber membrane is modified with or contains active agents. In certain embodiments, the 2D nanofiber membrane is modified with or contains active agents (e.g., antimicrobial, anti-infective, anti-adhesion, anti-fibrotic agents, or other agents which would be beneficial at the interface between the 2D nanofiber membrane and the intraabdominal viscera).

The 2D nanofiber membrane and/or 3D expanded nanofiber membrane of the instant invention may comprise or encapsulate at least one agent, particularly a bioactive agent such as a biologic, drug or therapeutic agent (e.g., analgesic, growth factor, anti-inflammatory, signaling molecule, cytokine, antimicrobial (e.g., antibacterial, antibiotic, antiviral, and/or antifungal), anti-infective, anti-fibrotic, anti-adhesive, blood clotting agent, factor, or protein, etc.). In a particular embodiment, the agent is hydrophilic. The agent may be added to the nanofiber membranes during synthesis and/or after synthesis. The agent may be conjugated to the nanofiber membrane and/or coating material, encapsulated by the nanofiber structure, and/or coated on the nanofiber structure (e.g., with, underneath, and/or on top of the coating that enhances the nanofiber membrane's ability to absorb fluids). In a particular embodiment, the agent is not directly conjugated to the nanofiber membrane (e.g., encapsulated). In a particular embodiment, the agent is conjugated or linked to the nanofiber membrane (e.g., surface conjugation or coating).

Biologics include but are not limited to proteins, peptides, antibodies, antibody fragments, DNA, RNA, and other known biologic substances, particularly those that have therapeutic use. In a particular embodiment, the agent is a drug or therapeutic agent (e.g., a small molecule) (e.g., analgesic, growth factor, anti-inflammatory, signaling molecule, cytokine, antimicrobial (e.g., antibacterial, antibiotic, antiviral, and/or antifungal), blood clotting agent, factor, or protein, pain medications (e.g., anesthetics), etc.). In a particular embodiment, the agent enhances tissue regeneration, tissue growth, and wound healing (e.g., growth factors). In a particular embodiment, the agent treats/prevents infections (e.g., antimicrobials such as antibacterials, antivirals and/or antifungals). In a particular embodiment, the agent is an antimicrobial, particularly an antibacterial. In a particular embodiment, the agent enhances wound healing and/or enhances tissue regeneration (e.g., bone, tendon, cartilage, skin, nerve, and/or blood vessel). Such agents include, for example, growth factors, cytokines, chemokines, immunomodulating compounds, and small molecules. Growth factors include, without limitation: platelet derived growth factors (PDGF), vascular endothelial growth factors (VEGF), epidermal growth factors (EGF), fibroblast growth factors (FGF; e.g., basic fibroblast growth factor (bFGF)), insulin-like growth factors (IGF-1 and/or IGF-2), bone morphogenetic proteins (e.g., BMP-2, BMP-7, BMP-12, BMP-9; particularly BMP-2 fragments, peptides, and/or analogs thereof), transforming growth factors (e.g., TGFβ, TGFβ3), nerve growth factors (NGF), neurotrophic factors, stromal derived factor-1 (SDF-1), granulocyte-macrophage colony-stimulating factor (GM-CSF), granulocyte-colony stimulating factor (G-CSF), erythropoietin (EPO), glial cell-derived neurotrophic factors (GDNF), hepatocyte growth factors (HGF), keratinocyte growth factors (KGF), and/or growth factor mimicking peptides (e.g., VEGF mimicking peptides). Chemokines include, without limitation: CCL21, CCL22, CCL2, CCL3, CCL5, CCL7, CCL8, CCL13, CCL17, CXCL9, CXCL10, and CXCL11. Cytokines include without limitation IL-2 subfamily cytokines, interferon subfamily cytokines, IL-10 subfamily cytokines, IL-1, 1-18, IL-17, tumor necrosis factor, and transforming-growth factor beta superfamily cytokines. Examples of small molecule drugs/therapeutic agents include, without limitation, simvastatin, kartogenin, retinoic acid, paclitaxel, vitamins (e.g., vitamin D3), etc. In a particular embodiment, the agent is a blood clotting factor such as thrombin or fibrinogen.

In certain embodiments, the nanofibers and/or nanofiber membranes (2D and/or 3D) are coated with additional materials to enhance their properties. In a particular embodiment, the nanofibers and/or nanofiber membranes is coated with additional materials to enhance their properties. For example, the nanofibers and/or nanofiber membrane may be coated with proteins, collagen, fibronectin, collagen, a proteoglycans, elastin, or a glycosaminoglycans (e.g., hyaluronic acid, heparin, chondroitin sulfate, or keratan sulfate). In a particular embodiment, the nanofiber membrane (e.g., the 3D expanded nanofiber membrane) comprises a material that enhances the nanofiber structure's ability to absorb fluids, particularly aqueous solutions (e.g., blood). In a particular embodiment, the nanofibers comprise a polymer and the material which enhances the absorption properties. In a particular embodiment, the nanofibers and/or nanofiber membranes are coated with the material which enhances the absorption properties. The term “coat” refers to a layer of a substance/material on the surface of a membrane. Coatings may, but need not, also impregnate the nanofiber membrane. Further, while a coating may cover 100% of the nanofibers and/or nanofiber membrane, a coating may also cover less than 100% of the surface of the nanofibers and/or nanofiber membrane (e.g., at least about 75%, at least about 80%, at least about 85%, at least about 90%, at least about 95%, at least about 98%, or more the surface may be coated). Materials which enhance the absorption properties of the expanded nanofiber membranes include, without limitation: gelatin, alginate, chitosan, collagen, starch, pectin, cellulose, methylcellulose, sodium polyacrylate, starch-acrylonitrile co-polymers, other natural or synthetic hydrogels, and derivatives thereof (e.g., del Valle et al., Gels (2017) 3:27). In a particular embodiment, the material is a hydrogel (e.g., a polymer matrix able to retain water, particularly large amounts of water, in a swollen state). In a particular embodiment, the material is gelatin. In a particular embodiment, the expanded nanofiber structures are coated with about 0.05% to about 10% coating material (e.g., gelatin), particularly about 0.1% to about 10% coating material (e.g., gelatin) or about 0.1% to about 1% coating material (e.g., gelatin). In a particular embodiment, the material (e.g., hydrogel) and/or nanofiber membrane is crosslinked. In a particular embodiment, the nanofiber membranes of the instant invention are crosslinked (e.g., before or after expansion or without expansion). Crosslinking may be done using a variety of techniques including thermal crosslinking, chemical crosslinking, and photo-crosslinking. For example, the nanofiber structures of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457; Bigi, et al., Biomaterials (2002) 23:4827-4832; Zhang, et al., Biomacromolecules (2010) 11:1125-1132; incorporated herein by reference). The crosslinker may be a bifunctional, trifunctional, or multifunctional crosslinking reagent. In a particular embodiment, the crosslinker is glutaraldehyde.

Examples of hernia meshes include multi-layer devices that comprise two or more components chosen from a 2D nanofiber membrane, a 3D expanded membrane, and a strengthening or support membrane. In certain embodiments, the hernia mesh comprises one 2D nanofiber membrane, one strengthening or support membrane, and one 3D expanded nanofiber membrane. In certain embodiments, the hernia mesh comprises one 2D nanofiber membrane and one 3D expanded nanofiber membrane. In certain embodiments, the hernia mesh comprises one 3D expanded nanofiber membrane, one strengthening or support membrane, and a second 3D expanded nanofiber membrane. The 3D expanded membrane-strengthening or support membrane-3D expanded membrane may be used for placement within the layers of the abdominal wall (e.g., a retrorectus hernia repair), away from the viscera.

The multi-layers of the hernia mesh may be the same size or of different sizes from each other (e.g., in terms of surface area and/or shape). In certain embodiments, the 2D nanofiber membrane may be larger than the 3D expanded nanofiber membrane. For example, when viewed from the 3D expanded nanofiber membrane side, a margin of the 2D nanofiber membrane may be viewed around the 3D expanded nanofiber membrane. In certain embodiments, the margin may be from about 0 to about 10 mm, particularly about 0 to about 5 mm. When present, the strengthening or support membrane is the same size as the 2D nanofiber membrane and/or 3D expanded nanofiber membrane or a different size. In a particular embodiment, the strengthening or support membrane is the same size as the 3D expanded nanofiber membrane or slightly larger than the 3D expanded nanofiber membrane. In a particular embodiment, the strengthening or support membrane is the same size as the 2D nanofiber membrane or slightly smaller than the 2D nanofiber membrane.

The layers of the multi-layer hernia mesh may be of different thicknesses. Typically, the membranes will be less than 5 mm in thickness. In certain embodiments, the membranes are about 0.1 mm to about 5 mm in thickness, about 0.5 mm to about 5 mm in thickness, about 0.5 mm to about 3 mm in thickness, or about 0.5 mm to about 2 mm in thickness. In certain embodiments, the 3D expanded nanofiber membrane is about 1.5 to about 5 times as thick as the 2D nanofiber membrane, particularly about 2 to about 3 times as thick. In certain embodiments, the 3D expanded nanofiber membrane is at least 1.1 times, 1.5 times, or 2 times as thick as the 2D nanofiber membrane.

The layers of the multi-layer hernia mesh may comprise the same or different polymers. In certain embodiments, the 2D nanofiber membrane comprises the same polymer as the 3D expanded nanofiber membrane. As explained hereinabove, while the 2D nanofiber membrane and the 3D expanded nanofiber membrane may comprise the same polymer, the membranes may be treated differently and/or comprise different agents and/or modifications.

The layers/membranes of the multi-layer hernia mesh may be bonded together by any means. Each of the membranes may be bonded together by the same means or by different means. In certain embodiments, at least the 2D nanofiber membrane and the 3D expanded nanofiber membrane are bonded together. In certain embodiments, the membranes are bonded only at the edge or border of the membrane. In certain embodiment, the membranes are bonded across the majority of or the entirety of the surface of the membrane. In certain embodiments, the membranes are bonded together by a biologically compatible means. For example, the membranes may be bonded together by a thermal bond, pressure bond, sutures, fasteners, glue, sealant, or any other suitable attachment mechanism. The bonding means may be permanent and/or non-biodegradable or may be biodegradable. In certain embodiments, the membranes may be stitched together or sutured. In certain embodiment, the membranes are bonded together with a biocompatible glue or biocompatible sealant. In a particular embodiment, the membranes are bonded together with a biocompatible glue.

The hernia meshes of the instant invention may also be sterilized. For example, the hernia meshes can be sterilized using various methods (e.g., by treating with ethylene oxide gas, gamma irradiation, or 70% ethanol).

The instant application encompasses the hernia meshes synthesized by the methods of the instant invention. Compositions comprising the hernia meshes synthesized by the methods of the instant invention and at least one pharmaceutically or biologically acceptable carrier are also encompassed by the instant invention.

In accordance with the instant invention, the hernia meshes may be used in treating, preventing, and/or inhibiting a hernia in a subject. For example, the hernia meshes can be used to induce, improve, or enhance hernia healing and/or prevent and/or inhibit hernia formation. In accordance with the instant invention, methods for treating, preventing, and/or inhibiting a hernia and/or inducing and/or improving/enhancing hernia healing in a subject are also provided. The hernia can be any hernia type, in any location of the body, and of any complexity. For example, the multi-layer hernia mesh of the instant invention can be used to treat complicated hernias, uncomplicated hernias, groin hernias, infected abdominal wound dehiscence, hiatal hernia repair, complicated abdominal wall defects, or hernia prophylaxis in high risk patients. In a particular embodiment, the hernia is a complicated hernia such as one involving contaminated/infected abdominal wounds. In a particular embodiment, the hernia is a retrorectus hernia. In a particular embodiment, the hernia mesh is used in an intraperitoneal placement of mesh (IPOM). In a particular embodiment, the hernia is one for which permanent mesh is contraindicated or not preferred. In certain embodiments, the hernia is in the abdominal cavity. In certain embodiments, the hernia mesh is for incisional support and/or prevention of hernia formation.

The methods of the instant invention comprise administering, implanting (e.g., surgically (e.g., via sutures)) or applying a hernia mesh of the instant invention to the subject (e.g., at a hernia). In a particular embodiment, the method comprises administering or implanting a hernia mesh comprising an agent as described hereinabove. In a particular embodiment, the method comprises administering or implanting a hernia mesh to the subject and an agent as described hereinabove (i.e., the agent is not contained within the nanofiber structure). When administered separately, the hernia mesh may be administered simultaneously and/or sequentially with the agent. The methods may comprise the administration of one or more hernia meshes. When more than one hernia mesh is administered, the hernia mesh may be administered simultaneously and/or sequentially.

The hernia mesh of the instant invention may be used for the management of hernia disease in obese patients. Specifically, the hernia mesh of the instant invention may be used in the treatment of complicated hernia and in the primary prevention (prophylaxis) of hernia, which both have an elevated risk and incidence in obese patients (Sauerland, et al. (2004) Hernia 8(1):42-46; Newcomb, et al. (2008) Hernia 12(5):465-469; Samson, et al. (2005) Plast. Reconstr. Surg. 116(2):523-527). In addition, the hernia mesh of the instant invention may be used to reinforce surgical incisions which are at risk for hernia development (e.g., hernia prophylaxis (Borab, et al. (2017) Surgery 161(4):1149-1163)), particularly those in obese patients (Sauerland, et al. (2004) Hernia 8(1):42-46).

### Definitions

The singular forms “a,” “an,” and “the” include plural referents unless the context clearly dictates otherwise.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In a particular embodiment, hydrophobic polymers may have aqueous solubility less than about 1% wt. at 37° C. In a particular embodiment, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, an “anti-inflammatory agent” refers to compounds for the treatment or inhibition of inflammation. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, acetaminophen, glucocorticoids, steroids, beta-agonists, anticholinergic agents, methyl xanthines, gold injections (e.g., sodium aurothiomalate), sulphasalazine, and dapsone.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain).

As used herein, the term “small molecule” refers to a substance or compound that has a relatively low molecular weight (e.g., less than 2,000). Typically, small molecules are organic, but are not proteins, polypeptides, or nucleic acids.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. A “photocrosslinker” refers to a molecule capable of forming a covalent linkage between compounds after photoinduction (e.g., exposure to electromagnetic radiation in the visible and near-visible range). Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

The following example illustrates certain embodiments of the invention. It is not intended to limit the invention in any way.

### Example

Expanded synthetic nanofiber matrices for use as a hernia mesh were synthesized (PCT/US2015/052858; Jiang, et al. (2015) ACS Biomater. Sci. Engineering, 1(10):991-1001; Jiang, et al. (2016) Adv. Healthcare Mater., 5(23):2993-3003). The synthesized hernia meshes provide the combination of mechanical strength, handling characteristics, slow in vivo degradation rate, and economy of production, making the hernia meshes superior to currently available synthetic hernia meshes.

Nanofiber mesh was electrospun from 20% (w/v) polycaprolactone/polylactide, PCL/PLA (50:50 blend) in 4:1 dichloromethane:dimethylformamide as described (Xie, et al. (2012) Adv. Healthcare Mater. 1(5):674-678; Xie, et al. (2012) Macromol. Biosci., 12(10):1336-1341; Xie, et al. (2013) Acta Biomater., 9(3):5698-5707). The nanofiber mesh was then trimmed to the desired size (10×10 cm). The thickness of the electrospun PCL/PLA nanofiber sheets was expanded several-fold using a gas-foaming technique—either a sodium borohydride solution or a CO₂ subcritical fluid and reduced pressure—to a final thickness of 2-3 mm (Jiang, et al. (2015) ACS Biomater. Sci. Engineering, 1(10):991-1001; Jiang, et al. (2016) Adv. Healthcare Mater., 5(23):2993-3003). The expanded mesh was then coated with gelatin and crosslinked with glutaraldehyde.

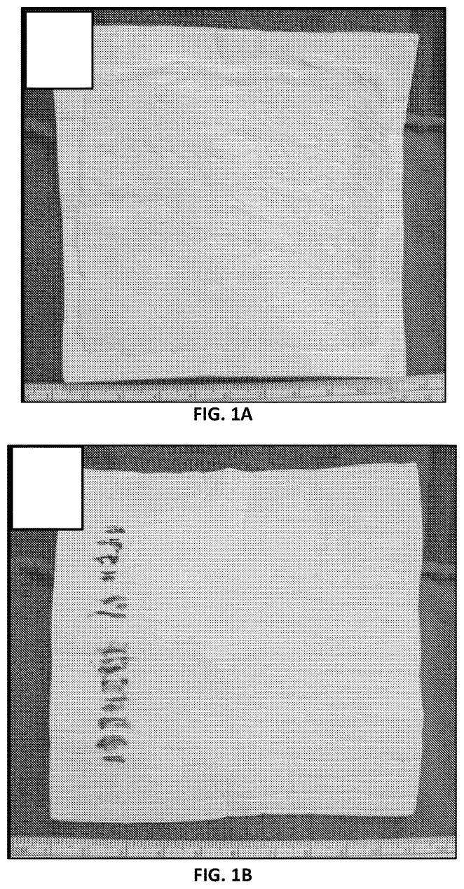

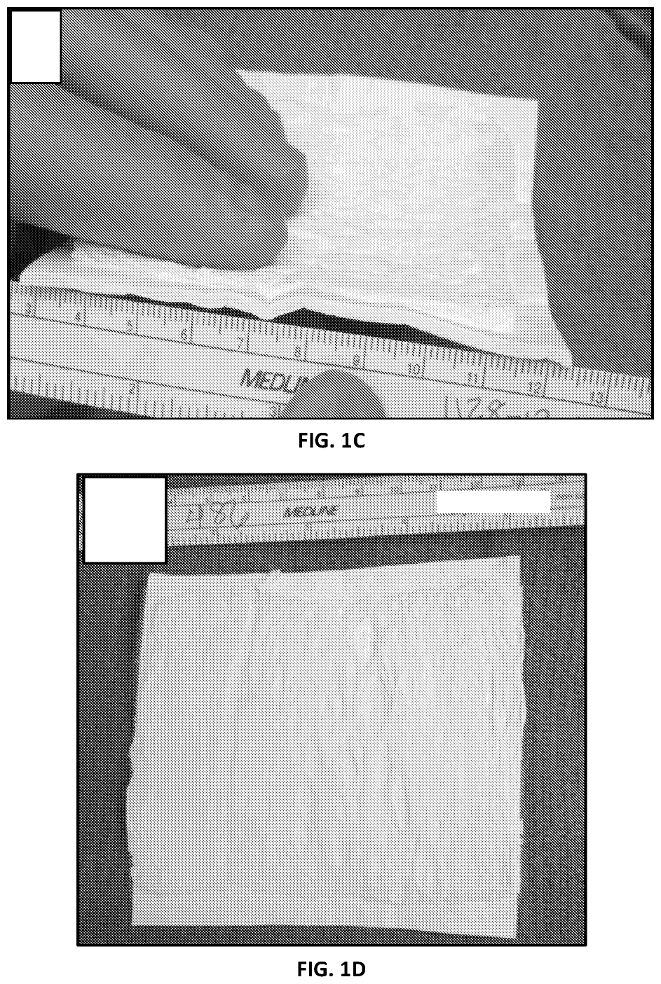

A fully resorbable mesh (e.g., for shorter term incision support) and a partially resorbable mesh (e.g., for longer term incision support) were generated. The fully resorbable (2D/3D) mesh comprises a non-expanded (2D) layer of the PCL/PLA electrospun nanofiber mesh bonded to a gas-expanded (3D) layer of the PCL/PLA electrospun nanofiber mesh by a biocompatible glue (see, FIGS. 1A-1C). The partially resorbable (2D/PPE/3D) mesh comprises the 2D/3D mesh with a layer of lightweight polypropylene (PPE; uncoated Atrium® ProLite® Ultra) sandwiched in between the layers (FIG. 1D).

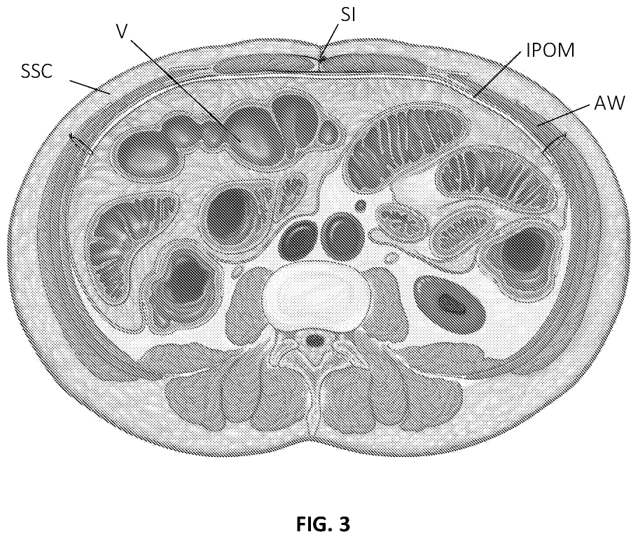

In hernia surgery, mesh often is placed on the underside of the abdominal wall as a reinforcement of incisional closure. This technique is referred to IPOM (intraperitoneal placement of mesh (Kockerling, et al. (2019) Surg. Endosc., 33(10):3361-3369)), since the mesh actually is within the peritoneal cavity with one side of the mesh facing the abdominal wall and the other side facing the viscera (i.e., intestines, other internal organs) (FIG. 3). The IPOM technique is useful for reinforcing or buttressing an incisional closure which is at risk for disruption, which is particularly relevant for infected incisions or in obese patients. In order for this technique to work best, the side of the mesh facing the abdominal wall should promote tissue ingrowth so that the mesh incorporates into the wall. However, the side of the mesh facing the viscera should resist tissue ingrowth and adhesion formation, as these sequelae are associated with a risk of mesh erosion into the intestines, which is a dreaded complication associated with the IPOM technique (Van Hoef, et al. (2019) Hernia 23(5):915-925). The 2D non-porous layer of the multi-layer mesh construction of the instant invention is in contact with the viscera after IPOM hernia repair, since the 2D non-porous layer will minimize adhesion formation and tissue ingrowth (Aydemir Sezer, et al. (2019) Mater. Sci. Eng. C Mater. Biol. Appl., 99:1141-1152; Balique, et al. (2005) Hernia 9(1):68-74). The 3D expanded layer is in contact with the abdominal wall to encourage tissue ingrowth (Jiang, et al. (2016) Adv. Healthcare Mater., 5(23):2993-3003; Chen, et al. (2018) Biomaterials 179:46-59; Chen, et al. (2020) Appl. Phys. Rev., 7(2):021406; Chen, et al. (2020) J. Mater. Chem. B, 8(17):3733-3746; Chen, et al. (2017) Nanomedicine (Lond) 12(11):1335-1352; Chen, et al. (2020) Adv. Mater., 2020:e2003754; Chen, et al. (2020) Acta Biomater., 108:153-167; Jiang, et al. (2018) Acta Biomater., 68:237-248).

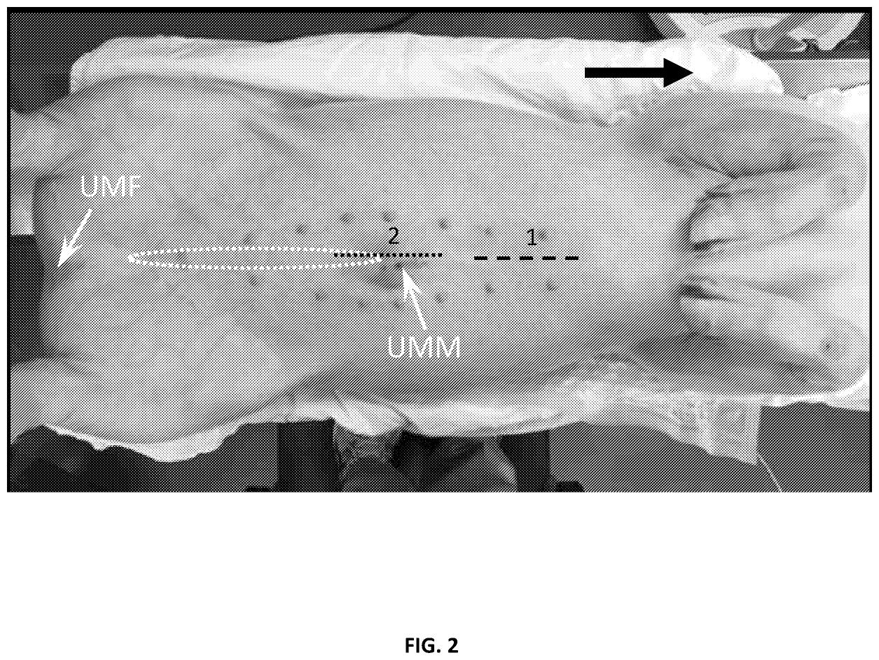

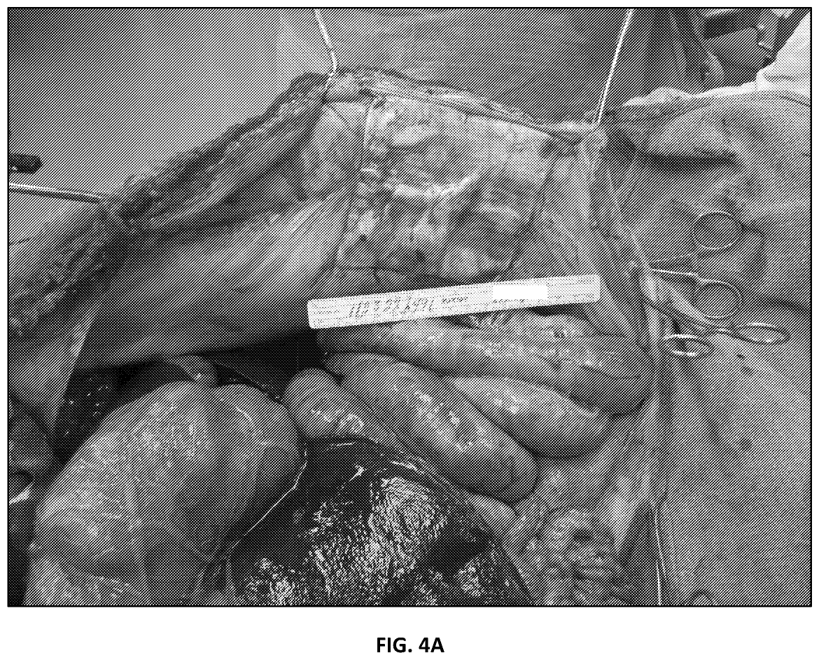

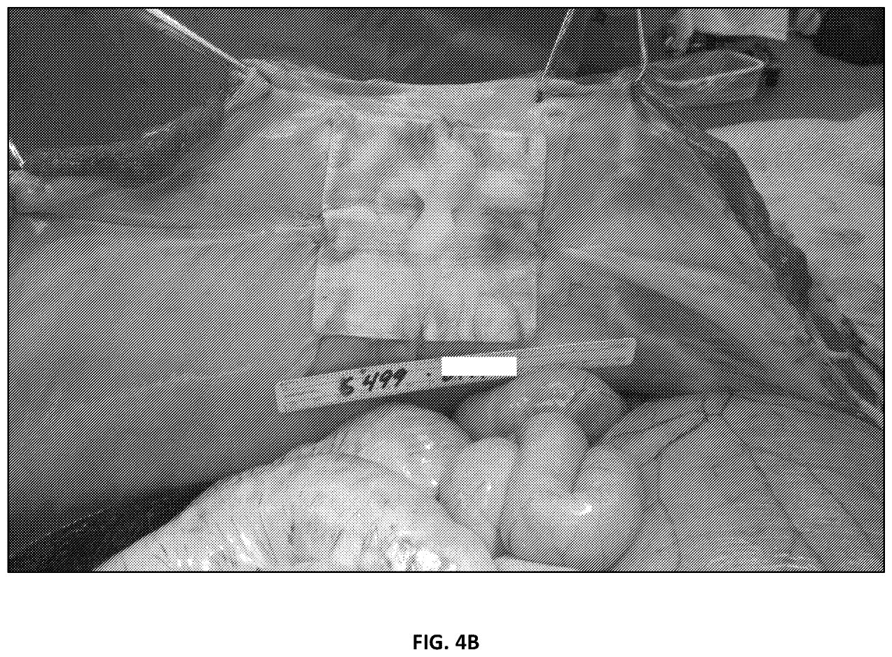

The fully resorbable mesh (n=4) and the partially resorbable mesh (n=5) were tested in a porcine survival model of abdominal wall hernia repair (Deeken, et al. (2011) J. Am. Coll. Surg., 212(5):880-888; Cobb, et al. (2006) J. Surg. Res., 136(1):1-7). Control groups (commercial biologic mesh (Biodesign®, Cook Medical), commercial polypropylene mesh (Proceed®; Ethicon), and no mesh (suture closure only)) with a similar number of pigs were also tested. Specifically, an intraperitoneal underlay placement of a 10×10 cm sheet of mesh (anchored with 0-Vicryl® sutures around the perimeter) to reinforce a ventral linea alba incision in female domestic pigs (3 months, 35 kg). Since a clinically important endpoint in this study is the degree of intestinal adhesions to the mesh, the midline incision was relocated more inferiorly to a central midline incision (dashed line no. 2 in FIG. 2). It was determined that mesh implanted through a central midline incision was 100% in contact with intestines. Pigs were euthanized at 90 days. No significant systemic toxicity was observed for the fully resorbable mesh and the partially resorbable mesh. Significantly, minimal to no adhesions was observed for the fully resorbable mesh and the partially resorbable mesh. As seen in FIGS. 4A and 4B, the fully resorbable mesh and the partially resorbable mesh remained intact at 90 days.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A hernia mesh comprising multiple layers selected from 2D nanofiber membranes, 3D expanded nanofiber membranes, and/or strengthening or support membranes.

2. The hernia mesh of claim 1, wherein said hernia mesh comprises a 2D nanofiber membrane and a 3D expanded nanofiber membrane.

3. The hernia mesh of claim 2, wherein said hernia mesh further comprises a strengthening or support membrane.

4. The hernia mesh of claim 1, wherein the 2D nanofiber membranes and the 3D expanded nanofiber membranes comprise a polymer selected from the group consisting of polymethacrylate, poly vinyl phenol, polyvinylchloride, cellulose, polyvinyl alcohol, polyacrylamide, poly(lactic-co-glycolic) acid (PLGA), collagen, polycaprolactone (PCL), polyurethanes, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene gricol, polyethylene glycol, poly(ethylene-co-vinyl alcohol), polyacrylnitrile, polyvinyl pyrrolidone, polymetha-phenylene isophthalamide, gelatin, alginate, chitosan, starch, pectin, cellulose, methylcellulose, sodium polyacrylate, starch-acrylonitrile co-polymers, and combinations of two or more polymers.

5. The hernia mesh of claim 4, wherein the 2D nanofiber membranes and the 3D expanded nanofiber membranes comprise polycaprolactone (PCL) and polylactide (PLA).

6. The hernia mesh of claim 1, wherein the 2D nanofiber membranes and/or the 3D expanded nanofiber membranes comprise electrospun nanofibers.

7. The hernia mesh of claim 1, wherein the 3D expanded nanofiber membrane is manufactured using a gas expansion method.

8. The hernia mesh of claim 1, wherein said hernia mesh comprises one layer of a 3D expanded nanofiber membrane, one layer of a strengthening or support membrane, and a second layer of a 3D expanded nanofiber membrane.

9. The hernia mesh of claim 1, wherein said 2D nanofiber membrane and/or 3D expanded nanofiber membrane comprises one or more active agents.

10. The hernia mesh of claim 9, wherein said active agents are selected from the group consisting of antimicrobial agents, chemokines, cytokines, and growth factors.

11. The hernia mesh of claim 1, wherein the surface of the 3D expanded nanofiber membrane comprises extra cellular matrix mimetic materials, a hydrogel, antimicrobial agents and/or growth factors.

12. The hernia mesh of claim 1, wherein the 3D expanded nanofiber membrane is coated with gelatin and crosslinked.

13. A method of treating, preventing, and/or inhibiting a hernia in a subject in need thereof, said method comprising administering to said subject a hernia mesh of claim 1.

14. A method for producing a hernia mesh comprising multiple layers, said method comprising
a) synthesizing a first 2D nanofiber membrane;
b) synthesizing a second 2D nanofiber membrane and expanding the second 2D nanofiber membrane to generate a 3D expanded nanofiber membrane, and
c) bonding said 2D nanofiber membrane and said 3D expanded nanofiber membrane, optionally with a strengthening or support membrane, thereby producing said hernia mesh comprising multiple layers.
